(19) United States
(12) Patent Application Publication
(10) Pub. No.: US 2026/0243649 A1
(43) Pub. Date: Aug. 20, 2026

(54) DEVICE AND METHOD FOR GROUTING TEST IN MINING-INDUCED FRACTURED ROCK MASSES WITH REAL-TIME SYNCHRONOUS PERMEABILITY MEASUREMENT

(71) Applicant: CHINA UNIVERSITY OF MINING AND TECHNOLOGY, Xuzhou City (CN)

(72) Inventors: Dan MA, Xuzhou City (CN); Jixiong ZHANG, Xuzhou City (CN); Hongyu DUAN, Xuzhou City (CN); Zhisong MA, Xuzhou City (CN); Xuefeng GAO, Xuzhou City (CN); Qiang LI, Xuzhou City (CN); Chuanjiu ZHANG, Xuzhou City (CN); Wenxin LI, Xuzhou City (CN); Baoli WANG, Xuzhou City (CN)

(21) Appl. No.: 19/531,905

(22) Filed: Feb. 6, 2026

(30) Foreign Application Priority Data
Feb. 19, 2025 (CN) 202510181953.0

(51) Int. Cl. G01N 15/08; G01N 33/24

(52) U.S. Cl. CPC G01N 15/0826; G01N 33/24

(57) ABSTRACT

A device and a method for grouting test in mining-induced fractured rock masses with real-time synchronous permeability measurement are provided. The device includes a base arranged in a pressure applying device with a chamber; a gas intake part arranged at the bottom of the chamber bottom; a gas exhaust part arranged at the top of the chamber, the gas exhaust part is arranged in a piston, the piston is slidably arranged in the chamber; a grouting part wrapped around the side wall of the sample, the bottom of the grouting part is fixedly connected to the base, and a liquid outlet end is in communication with the chamber; and a displacement sensor for detecting a distance between the pressure applying end and the top of the chamber.

### CROSS-REFERENCE TO RELATED APPLICATIONS

This application claims priority to Chinese Patent Application No. 202510181953.0, filed on Feb. 19, 2025, the contents of which are hereby incorporated by reference.

### TECHNICAL FIELD

The present disclosure belongs to the technical field of grouting and filling for mining-induced fractured rock masses, and particularly relates to a device and a method for grouting test in mining-induced fractured rock masses with real-time synchronous permeability measurement.

### BACKGROUND

During coal seam mining, affected by mining activities, fractures continuously develop inside the overlying rock strata, and the porosity and permeability increase. When the mining-induced fractures are connected upward to the aquifer, water inrush accidents occur, resulting in casualties and property losses. Grouting technology may effectively fill the mining-induced fractures in the overlying rock to form a water-resisting rock stratum, thereby preventing water inrush accidents. During this process, the permeability of the fractured rock mass continuously decreases under the filling effect of the grout, but existing methods may not reproduce and real-time synchronously measure this process.

Therefore, there is an urgent need for a device and a method for grouting test in mining-induced fractured rock masses with real-time synchronous permeability measurement.

### SUMMARY

The present disclosure aims to provide a device and a method for grouting test in mining-induced fractured rock masses with real-time synchronous permeability measurement to solve the above-mentioned problems.

To achieve the above objective, the present disclosure provides the following technical solution.

A device for grouting test in mining-induced fractured rock masses with real-time synchronous permeability measurement is provided, the device includes:
a base arranged in a pressure applying device, where the base is provided with a chamber for storing a sample;
a gas intake part arranged at a bottom of the chamber, where a gas outlet end of the gas intake part is in contact with a bottom of the sample, a gas inlet end of the gas intake part is located outside the base, and the gas intake part is configured for communicating with a gas source and injecting high-pressure gas into the chamber;
a gas exhaust part arranged at a top of the chamber, where a gas inlet end of the gas exhaust part is in contact with a top of the sample, the gas exhaust part is arranged in a piston, the piston is slidably arranged in the chamber, a gas outlet end of the gas exhaust part is located outside the piston, and a top of the piston is mounted on a pressure applying end of the pressure applying device;
a grouting part wrapped around a side wall of the sample, where a bottom of the grouting part is fixedly connected to the base, and a liquid outlet end of the grouting part is in communication with the chamber for injecting grout into the chamber; and
a displacement sensor configured for detecting a height of the piston exceeding a top of the chamber to obtain a height of the sample.

According to the above device for grouting test in mining-induced fractured rock masses with real-time synchronous permeability measurement, the grouting part includes:
an inner grouting cylinder, where a bottom of the inner grouting cylinder is axially connected to the base, the chamber is arranged in the inner grouting cylinder, and multiple grouting holes are uniformly formed on a side wall of the inner grouting cylinder for communicating with the chamber;
an outer cylinder coaxially arranged outside the inner grouting cylinder, where a bottom of the outer cylinder is fixedly connected to a top of the base, and a top of the outer cylinder is fixedly connected to a top of the inner grouting cylinder;
a grout temporary storage cavity enclosed by an inner wall of the outer cylinder and an outer wall of the inner grouting cylinder, where the grout temporary storage cavity is in communication with liquid inlet ends of the grouting holes; and
a grouting port in communication with the grout temporary storage cavity, where the grouting port is formed on the outer cylinder.

According to the above device for grouting test in mining-induced fractured rock masses with real-time synchronous permeability measurement, the gas intake part includes:
a first honeycomb porous gas cavity arranged at the bottom of the chamber, where a gas inlet end of the first honeycomb porous gas cavity is in communication with a gas outlet end of a gas intake pipeline, the gas intake pipeline is embedded in the base, a gas inlet of the gas intake pipeline is arranged on a side wall of the base, and the gas inlet is configured for connecting with the gas source; and
a gas-permeable fine gauze laid between a gas outlet end of the first honeycomb porous gas cavity and the sample.

According to the above device for grouting test in mining-induced fractured rock masses with real-time synchronous permeability measurement, the gas exhaust part includes:
a second honeycomb porous gas cavity slidably arranged in the chamber, where the second honeycomb porous gas cavity is fixedly connected to a bottom of the piston, a gas inlet end of the second honeycomb porous gas cavity is in contact with and in communication with a top of the sample, a gas outlet end of the second honeycomb porous gas cavity is in communication with a gas inlet end of a gas exhaust pipeline, the gas exhaust pipeline is embedded in the piston, and a gas outlet of the gas exhaust pipeline is formed on a side wall of the piston.

According to the above device for grouting test in mining-induced fractured rock masses with real-time synchronous permeability measurement, the displacement sensor includes:
a linear variable differential transformer, where a fixed end of the linear variable differential transformer is fixedly connected to the top of the inner grouting cylinder, and a movable end of the linear variable differential transformer is fixedly connected to the pressure applying end of the pressure applying device.

According to the above device for grouting test in mining-induced fractured rock masses with real-time synchronous permeability measurement, the pressure applying device includes:
an axial displacement control device, where the base is arranged in the axial displacement control device, and a movable end of the axial displacement control device is fixedly connected to the top of the piston; and
where the movable end of the linear variable differential transformer is fixedly connected to the movable end of the axial displacement control device.

A method for grouting test in mining-induced fractured rock masses with real-time synchronous permeability measurement is provided, and is based on the above device for grouting test in mining-induced fractured rock masses with real-time synchronous permeability measurement. The method includes the following steps:
filling the sample into the chamber, and controlling the piston to be in contact with the sample;
driving the pressure applying end of the pressure applying device to drive the piston to press down the sample, adjusting a porosity of the sample, and detecting a displacement of the pressure applying end of the pressure applying device through the displacement sensor;
after reaching a target initial porosity, relieving pressure to a stable value and keeping a loading pressure of the pressure applying end of the pressure applying device unchanged, starting the gas source and the grouting mechanism to inject the grout into the chamber, and starting a seepage grouting test;
controlling the grouting part to inject the grout into the grouting mechanism, and enabling the grout to be injected into the chamber;
communicating the gas source with the gas intake part, and controlling the gas source to inject the high-pressure gas into the chamber through the gas intake part;
communicating the gas collection device with the gas exhaust part;
obtaining gas inlet pressure data based on a gas inlet flow rate of the gas intake part;
obtaining gas outlet pressure data based on a gas outlet flow rate of the gas exhaust part;
obtaining grouting pressure data of the grouting part;
obtaining the permeability and nonlinear seepage factor based on the gas inlet flow rate, the gas inlet pressure data, the gas outlet flow rate, the gas outlet pressure data, and the grouting pressure data.

Compared with the prior art, the present disclosure has the following advantages and technical effects.

By providing axial stress to the sample, the present disclosure may change different initial porosities of the mining-induced fractured rock mass on the one hand, and may be configured to provide axial stress on the other hand; nitrogen may be configured as the seepage fluid, which may reduce the mutual influence with the grout during the grouting process. The gas flows from bottom to top, which may overcome the influence of the gas's own gravity; by monitoring parameters such as grout flow rate and gas flow rate during the grouting test, the permeability change law of the mining-induced fractured rock mass during the grouting process may be obtained after computer processing.

On the premise of keeping the grouting pressure and the gas pressure at the gas inlet unchanged, the present disclosure may obtain the permeability change characteristics of the mining-induced fractured rock mass under different axial loading pressures by changing the axial load; on the premise of keeping the axial load and the grouting pressure unchanged, the permeability change characteristics of the mining-induced fractured rock mass under different pore pressures may be obtained by changing the gas pressure at the gas inlet. On the premise of keeping the axial load and the gas pressure at the gas inlet unchanged, the permeability change characteristics of the mining-induced fractured rock mass under different grouting pressures may be obtained by changing the grouting pressure.

The present disclosure may realize the permeability change characteristics of the mining-induced fractured rock mass under different initial porosities, different continuous gradation conditions, and different grout property conditions.

### BRIEF DESCRIPTION OF THE DRAWINGS

In order to more clearly illustrate the embodiments of the present disclosure or the technical solutions in the prior art, the drawings required in the embodiments will be briefly introduced below. Apparently, the drawings in the following description are only some embodiments of the present disclosure, and for those of ordinary skill in the art, other drawings may be obtained according to these drawings without creative effort.

FIG. 1 is a schematic structural diagram of the present disclosure.

FIG. 2 is a schematic structural diagram of the second honeycomb porous gas cavity of the present disclosure.

FIG. 3 is a schematic structural diagram of the grouting part of the present disclosure.

FIG. 4 is a graph showing the change law of permeability of the mining-induced fractured rock mass with time during the grouting process of the present disclosure.

FIG. 5 is a graph showing the change law of the nonlinear seepage factor of the mining-induced fractured rock mass with time during the grouting process of the present disclosure.

FIG. 6 is a flow chart of a method for grouting test in mining-induced fractured rock masses with real-time synchronous permeability measurement.

### DETAILED DESCRIPTION OF THE EMBODIMENTS

The technical solutions in the embodiments of the present disclosure will be clearly and completely described below with reference to the drawings in the embodiments of the present disclosure. Apparently, the described embodiments are only a part of the embodiments of the present disclosure, not all of them. Based on the embodiments of the present disclosure, all other embodiments obtained by those of ordinary skill in the art without creative efforts shall fall within the protection scope of the present disclosure.

In order to make the above objectives, features and advantages of the present disclosure more obvious and understandable, the present disclosure will be further described in detail below with reference to the drawings and specific embodiments.

### Embodiment 1

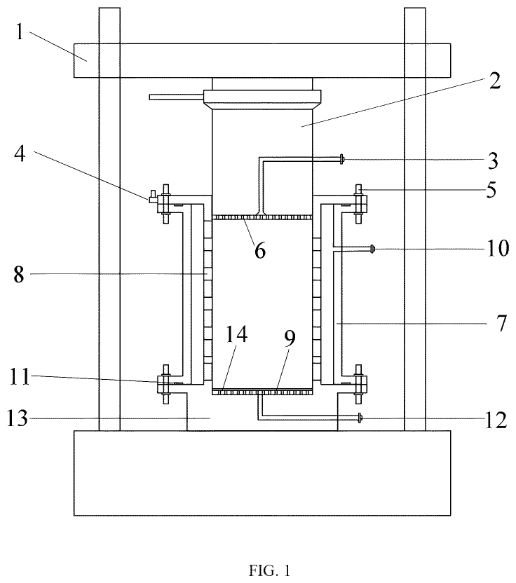

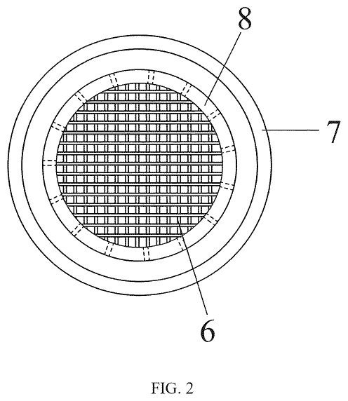

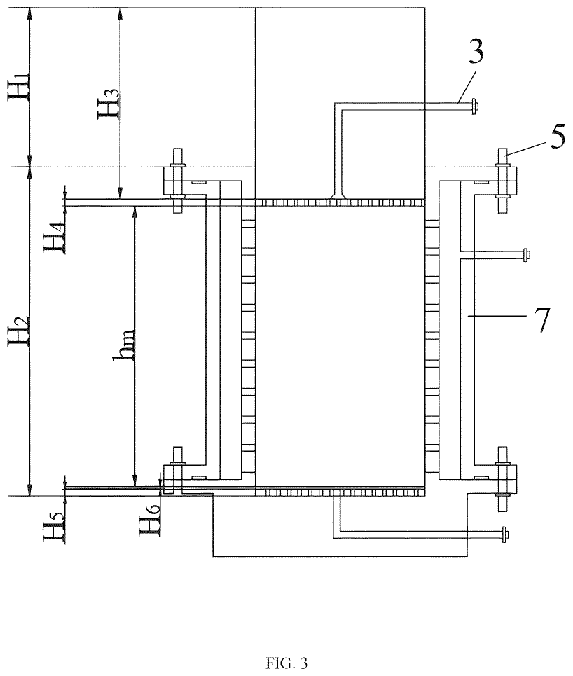

Referring to FIG. 1, FIG. 2, and FIG. 3, this embodiment discloses a device for grouting test in mining-induced fractured rock masses with real-time synchronous permeability measurement, which includes a base 13, a gas intake part, a gas exhaust part, a grouting part, and a displacement sensor.

The base 13 is arranged in a pressure applying device, where the base 13 is provided with a chamber for storing a sample.

The gas intake part is arranged at a bottom of the chamber, a gas outlet end of the gas intake part is in contact with a bottom of the sample, a gas inlet end of the gas intake part is located outside the base 13, and the gas intake part is configured for communicating with a gas source and injecting high-pressure gas into the chamber.

The gas exhaust part is arranged at a top of the chamber, a gas inlet end of the gas exhaust part is in contact with a top of the sample, the gas exhaust part is arranged in a piston 2, the piston 2 is slidably arranged in the chamber, a gas outlet end of the gas exhaust part is located outside the piston 2, and a top of the piston 2 is mounted on a pressure applying end of the pressure applying device.

The grouting part is wrapped around a side wall of the sample, where a bottom of the grouting part is fixedly connected to the base 13, and a liquid outlet end of the grouting part is in communication with the chamber for injecting grout into the chamber.

The displacement sensor is configured for detecting the height of the piston 2 exceeding a top of the chamber to obtain the height of the sample.

When in use, the sample is filled into the chamber, the piston 2 is placed at the top of the chamber to be in contact with the sample, and the piston 2 is connected with the pressure applying end of the pressure applying device; the grouting mechanism is in communication with the grouting part, the gas source is in communication with the gas intake part, the gas collection device (such as a gas collector or a gas collecting tank) is in communication with the gas exhaust part, the data acquisition device is arranged and the valve is installed, the pressure applying end of the pressure applying device is driven to drive the piston 2 to press down the sample to adjust the porosity of the sample. The loading pressure of the pressure applying end of the pressure applying device is kept unchanged, the gas source and the grouting mechanism are started to inject the grout into the sample, and the seepage grouting test is started. The data obtained by the computer are analyzed and processed to obtain the permeability of the mining-induced fractured rock mass under the grouting condition. By providing axial stress to the sample, the present disclosure may change different initial porosities of the mining-induced fractured rock mass on the one hand, and may be configured to provide axial stress on the other hand. Nitrogen may be used as the seepage fluid, which may reduce the mutual influence with the grout during the grouting process. The gas flows from bottom to top, which may overcome the influence of the gas's own gravity. By monitoring parameters such as the grout flow rate and gas flow rate during the grouting test, the permeability change law of the mining-induced fractured rock mass during the grouting process may be obtained after computer processing.

On the premise of keeping the grouting pressure and the gas pressure at the gas inlet unchanged, the present disclosure may obtain the permeability change characteristics of the mining-induced fractured rock mass under different axial loading pressures by changing the axial load. On the premise of keeping the axial load and the grouting pressure unchanged, the permeability change characteristics of the mining-induced fractured rock mass under different pore pressures may be obtained by changing the gas pressure at the gas inlet. On the premise of keeping the axial load and the gas pressure at the gas inlet unchanged, the permeability change characteristics of the mining-induced fractured rock mass under different grouting pressures may be obtained by changing the grouting pressure.

The present disclosure may realize the permeability change characteristics of the mining-induced fractured rock mass under different initial porosities, different continuous gradation conditions, and different grout property conditions.

As an optional implementation, the grouting part includes:
an inner grouting cylinder 8, where a bottom of the inner grouting cylinder 8 is axially connected to the base 13, the chamber is arranged in the inner grouting cylinder 8, and multiple grouting holes are uniformly formed on a side wall of the inner grouting cylinder 8 for communicating with the chamber;
an outer cylinder 7 coaxially arranged outside the inner grouting cylinder 8, where a bottom of the outer cylinder 7 is fixedly connected to a top of the base 13, and a top of the outer cylinder is fixedly connected to a top of the inner grouting cylinder 8;
a grout temporary storage cavity enclosed by an inner wall of the outer cylinder 7 and an outer wall of the inner grouting cylinder 8, where the grout temporary storage cavity is in communication with liquid inlet ends of the multiple grouting holes; and
a grouting port 10 in communication with the grout temporary storage cavity, where the grouting port 10 is formed on the outer cylinder 7.

The multiple grouting holes uniformly formed on the side wall of the inner grouting cylinder 8 are configured to ensure uniform grout injection.

Sealing rings 11 are arranged at the connection positions between the outer cylinder 7 and the inner grouting cylinder 8 to prevent grout from leaking out.

The grouting port 10 is connected to a grouting system, and the grouting system includes a grouting tank, a gas pump, a pressure stabilizing tank, a mud valve, a gas inlet valve, a pressure reducing valve, an exhaust pipe, an exhaust valve, a grouting hose, pressure gauges, a flow meter, and a grouting port. The gas pump, the pressure stabilizing tank, the grouting tank, and the grouting port 10 are connected in sequence; the grouting tank is provided with a mixer; the pressure reducing valve is arranged at the outlet of the pressure stabilizing tank, the gas inlet valve is arranged at the inlet of the pressure stabilizing tank, the exhaust pipe is arranged between the pressure reducing valve and the pressure stabilizing tank, and the exhaust pipe is provided with the exhaust valve; the mud valve is arranged at the outlet of the grouting tank; the pressure gauge is arranged at the inlet of the grouting tank, and the pressure gauge and the flow meter are arranged at the grouting port in sequence. The grouting system is the prior art, and will not be described in detail herein.

As an optional implementation, the gas intake part includes:
a first honeycomb porous gas cavity 9 arranged at the bottom of the chamber, where a gas inlet end of the first honeycomb porous gas cavity 9 is in communication with a gas outlet end of a gas intake pipeline, the gas intake pipeline is embedded in the base 13, a gas inlet 12 of the gas intake pipeline is arranged on a side wall of the base 13, and the gas inlet 12 is configured for connecting with the gas source; and
a gas-permeable fine gauze 14 laid between a gas outlet end of the first honeycomb porous gas cavity 9 and the sample.

The arrangement of the first honeycomb porous gas cavity 9 and the gas-permeable fine gauze 14 is configured to ensure uniform gas entry.

As an optional implementation, the gas exhaust part includes:
a second honeycomb porous gas cavity 6 slidably arranged in the chamber, where the second honeycomb porous gas cavity 6 is fixedly connected to a bottom of the piston 2, a gas inlet end of the second honeycomb porous gas cavity 6 is in contact with and in communication with a top of the sample, a gas outlet end of the second honeycomb porous gas cavity 6 is in communication with a gas inlet end of a gas exhaust pipeline, the gas exhaust pipeline is embedded in the piston 2, and a gas outlet 3 of the gas exhaust pipeline is formed on a side wall of the piston 2.

The arrangement of the second honeycomb porous gas cavity 6 is configured to ensure uniform gas discharge.

Both the gas inlet 12 and the gas outlet 3 are connected to a gas seepage system, and the gas seepage system includes a high-pressure nitrogen cylinder, a pressure reducing valve, a flow meter, a pressure sensor, a gas collection device, a gas inlet, a gas outlet, and a gas pipe. The gas inlet 12 is in communication with the high-pressure nitrogen cylinder, the gas outlet 3 is in communication with the gas collection device, the flow meter and the pressure reducing valve are arranged near one end of the gas inlet 12 to control the gas inlet flow rate and pressure, and the pressure sensor is arranged at the gas inlet 12 and the gas outlet 3.

The first honeycomb porous gas cavity 9 and the second honeycomb porous gas cavity 6 have the same structure.

As an optional implementation, the displacement sensor includes:
a linear variable differential transformer 4, where a fixed end of the linear variable differential transformer 4 is fixedly connected to the top of the inner grouting cylinder 8, and a movable end of the linear variable differential transformer 4 is fixedly connected to the pressure applying end of the pressure applying device.

As an optional implementation, the pressure applying device includes:
an axial displacement control device 1, where the base 13 is arranged in the axial displacement control device 1, and a movable end of the axial displacement control device 1 is fixedly connected to the top of the piston 2; and
where the movable end of the linear variable differential transformer 4 is fixedly connected to the movable end of the axial displacement control device 1.

The device further includes a data acquisition system, and the data acquisition system includes a data acquisition instrument and a computer. One end of the data acquisition instrument is electrically connected to the flow meters, the pressure gauges, the linear variable differential transformer 4, and the axial displacement control device 1 of the entire test system, and the other end is connected to the computer.

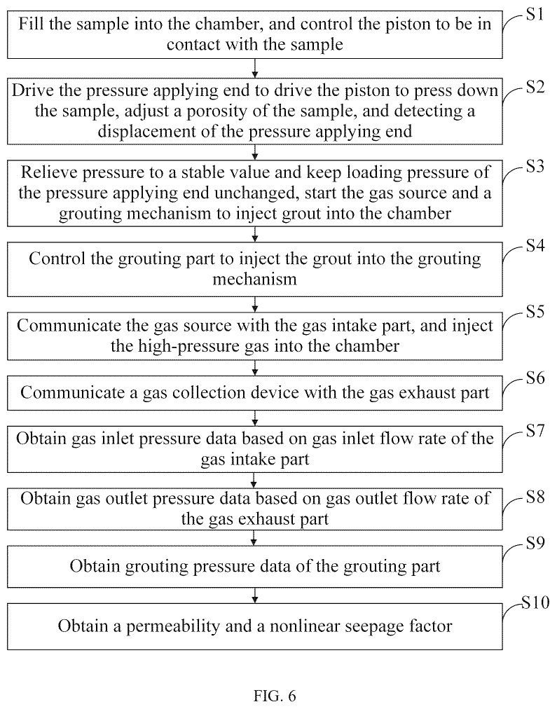

A method for grouting test in mining-induced fractured rock masses with real-time synchronous permeability measurement is provided, and is based on the above device for grouting test in mining-induced fractured rock masses with real-time synchronous permeability measurement. As shown in FIG. 6, the method includes the following steps:
S1: filling the sample into the chamber, and controlling the piston 2 to be in contact with the sample;
S2: driving the pressure applying end of the pressure applying device to drive the piston 2 to press down the sample, adjusting a porosity of the sample, and detecting a displacement of the pressure applying end of the pressure applying device through the displacement sensor;
S3: after reaching the target initial porosity, relieving pressure to a stable value and keeping the loading pressure of the pressure applying end of the pressure applying device unchanged, starting the gas source and the grouting mechanism to inject the grout into the chamber, and starting a seepage grouting test;
S4: controlling the grouting part to inject the grout into the grouting mechanism, and enabling the grout to be injected into the chamber;
S5: communicating the gas source with the gas intake part, and controlling the gas source to inject the high-pressure gas into the chamber through the gas intake part;
S6: communicating the gas collection device with the gas exhaust part;
S7: obtaining gas inlet pressure data based on the gas inlet flow rate of the gas intake part;
S8: obtaining gas outlet pressure data based on the gas outlet flow rate of the gas exhaust part;
S9: obtaining grouting pressure data of the grouting part; and
S10: obtaining the permeability and the nonlinear seepage factor based on the gas inlet flow rate, the gas inlet pressure data, the gas outlet flow rate, the gas outlet pressure data, and the grouting pressure data.

### Embodiment 2

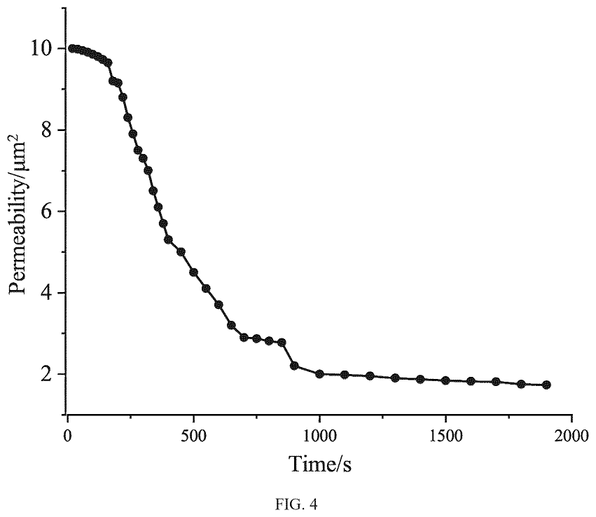

Referring to FIG. 4, this embodiment is a process of obtaining permeability using the device and method of Embodiment 1.

Step 1: setting the particle size range and continuous gradation conditions of the sample, loading the rock sample, calculating the mass of crushed stones with different particle sizes according to the Talbot gradation method, weighing them, and loading them into the chamber. Placing the piston 2 above the chamber, i.e., the sample, and making the piston 2 contact with the pressure applying end of the axial displacement control device 1.

Step 2: connecting the pipelines in place.

Step 3: installing the data acquisition equipment such as flow meters and pressure gauges for detecting the gas inlet flow rate, gas intake pressure, gas outlet flow rate, gas exhaust pressure, and grouting pressure data in place.

Step 4: adjusting the porosity of the sample: installing a linear variable differential transformer 4 on the test device, starting the axial displacement control device 1 to load the piston 2, and controlling the height of the sample to adjust the porosity of the sample.

The calculation process of porosity is as follows:

The calculation formula of the initial porosity Do after axial pressure loading is as follows:

$${ϕ}_{0} = \frac{{V}_{0} - {V}_{S}}{{V}_{0}} ;$$

where V₀ is the volume of the rock sample in the natural state after axial pressure loading; and V_{S} is the absolute dense volume of the rock sample.

The calculation formula of the absolute dense volume V_{S} of the rock sample is as follows:

$${V}_{S} = \frac{{m}_{s}}{{ρ}_{s}} ;$$

where mₛ is the mass of the rock sample; ρₛ is the mass density of the rock sample.

The height of the rock sample is hₘ:

$${h}_{m} = {H}_{1} + {H}_{2} - {H}_{3} - {H}_{4} - {H}_{5} - {H}_{6} ;$$

where H₁ is the measurement data of the linear variable differential transformer 4; H₂ is the height of the inner grouting cylinder 8; H₃ is the height of the piston 2; H₄ is the height of the second honeycomb porous gas cavity 6; H₅ is the height of the first honeycomb porous gas cavity 9; H₆ is the height of the gas-permeable fine gauze. During the test, H₁, H₂, H₃, H₄, H₅ and H₆ are all known or measurable, and the height of the gas-permeable fine gauze is small compared with other values and may be ignored. Start the axial displacement control device 1 to load the piston 2, the sample moves down by Δh, and the measurement data H₁ of the linear variable differential transformer 4 changes.

It is able to be known from the above that the calculation formula of the volume V₀ of the rock sample in the natural state after axial pressure loading is as follows:

$${V}_{0} = π {r}^{2} {h}_{m} ;$$

where r is the bottom radius of the inner grouting cylinder; hₘ is the height of the rock sample after axial pressure loading.

Then the corrected calculation formula of the initial porosity Do after axial pressure loading is as follows:

$${ϕ}_{0} = 1 - \frac{{m}_{s}}{π {r}^{2} {ρ}_{s} ( {H}_{1} + {H}_{2} - {H}_{3} - {H}_{4} - {H}_{5} - {H}_{6} )} ;$$

Step 5: starting the seepage grouting test.

First, the gas injection pipeline is opened, the axial loading pressure is kept unchanged, the gas valve of the high-pressure nitrogen gas tank in the gas injection pipeline is opened, the gas injection pressure is adjusted through connection with the pressure reducing valve, and the pressure reducing valve is adjusted until the gas pressure is stable and unchanged.

Then, the grouting pipeline is opened, a pressure reducing valve is set between the pressure stabilizing tank and the grouting tank. During the preparation stage, the pressure reducing valve is closed between the pressure stabilizing tank and the grouting tank and the exhaust valve of the pressure stabilizing tank, the gas inlet valve of the pressure stabilizing tank is opened, the gas pump is started until the pressure in the pressure stabilizing tank reaches the set pressure, the gas pump is closed. The gas inlet valve is closed, the pressure reducing valve and the mud valve are opened, and the pressure stabilizing tank delivers high-pressure gas into the grouting tank to squeeze the grout into the chamber through the mud valve. The grouting pressure is controlled to be unchanged by adjusting the pressure reducing valve and the mud valve.

The changes of grout flow rate and gas flow rate are measured during the grouting process; during this process, the flow meters arranged at the grouting port and gas inlet of the chamber, and the pressure sensors arranged at the gas inlet and gas outlet respectively monitor the grout flow rate, gas inlet flow rate, gas pressure at the gas inlet, and gas pressure at the gas outlet, and feed the data back to the data acquisition instrument.

When the grouting is started, the calculation formula of the porosity of the rock sample is:

$${ϕ}_{0} = \frac{{V}_{0} - {V}_{S} - {V}_{r}}{{V}_{0}} ;$$

where V₀ is the volume of the rock sample in the natural state after axial pressure loading; V_{S} is the absolute dense volume of the rock sample; and Vᵣ is the volume of the grout injected into the rock sample.

The volume Vᵣ of the grout injected into the rock sample may be obtained according to the flow meter at the grouting port, which satisfies the following relationship:

$${V}_{r} = {\int }_{0}^{{T}_{i}} Q ;$$

where

$${\int }_{0}^{{T}_{i}} Q$$

is the cumulative flow rate of the grout pipeline at time Tᵢ.

According to the measured gas flow rate data, the flow velocity of the seepage gas may be calculated as:

$$v = \frac{{Q}_{{T}_{I}}}{π {r}^{2}} ;$$

where Q_{T}_{_{I}} is the gas pipeline flow rate at the gas inlet; r is the bottom radius of the inner grouting cylinder; and v is the flow velocity of the seepage gas.

According to the Forchheimer theory, the fluid in the rock sample satisfies:

$$- Δ p = \frac{μ}{k} v + {ρ}_{ω} β {v}^{2} ;$$

where, μ is the fluid dynamic viscosity coefficient; k is the permeability of the mining-induced fractured rock mass; β is the non-Darcy factor; ρ_{w} is the fluid density; v is the fluid velocity;
and p is the fluid pressure.

According to a large number of test and simulation results, the empirical formula of the non-Darcy factor is as follows:

$$β = \frac{1.15 \times {10}^{- 6}}{k ϕ} ;$$

Based on the experimental measurement data, it is obtained that:

$$- Δ p = {p}_{in} - {p}_{out} ;$$

where pᵢₙ is the gas pressure at the gas inlet; and pₒᵤₜ is the gas pressure at the gas outlet.

Then the corrected permeability calculation formula is:

$$k = \frac{{Q}_{{T}_{i}}}{π {r}^{2} ( {p}_{in} - {p}_{out} )} ( μ + \frac{1.15 \times {10}^{- 6} {ρ}_{w} {h}_{m} {Q}_{{T}_{i}}}{π {r}^{2} {h}_{m} - {\int }_{0}^{{T}_{i}} Q - \frac{{m}_{s}}{{ρ}_{s}}} ) ;$$

where pᵢₙ is the gas pressure at the gas inlet; pₒᵤₜ is the gas pressure at the gas outlet; ρ_{w} is the fluid density; ms is the mass of the rock sample; ρₛ is the mass density of the rock sample; Q_{Ti} is the gas pipeline flow rate at the gas inlet; r is the bottom radius of the inner grouting cylinder; hₘ is the height of the rock sample after axial pressure loading; and

$${\int }_{0}^{{T}_{i}} Q$$

is the cumulative flow rate of the grout pipeline at time Tᵢ.

After the test is completed, the mud valve and the pressure reducing valve are closed, the exhaust valve is opened to discharge the gas in the pressure stabilizing tank, the grouting tank is cleaned, and the test is completed.

Step 6: analyzing data: the computer obtains the data stored in the recorder, uploads the gas pipeline flow rate at the gas inlet, the gas pressure at the inlet and outlet, the cumulative flow rate of the grout pipeline and other data to the computer, and after analyzing and processing the data, finally obtains the permeability of the mining-induced fractured rock mass at different times under the grouting condition.

### Embodiment 3

According to the permeability calculation process of Embodiment 2, a specific application example is provided. Red sandstone is selected as the rock sample for testing, crushed into particles by a rock crusher and screened with a sieve. The particle sizes are selected as a first particle size (0-5 millimeter), second particle size (5-10 millimeters), third particle size (10-15 millimeters), fourth particle size (15-20 millimeters), and fifth particle size (20-25 millimeters). The Talbot index is set to 1, 1200 grams (g) of red sandstone is taken with different particle sizes for mixing according to the above Talbot index for characterizing the particle size distribution, then the axial load is loaded to adjust the porosity of the sample, the initial porosity of the sample is set to 0.183, and the height of the sample is set to 130 mm. After starting the seepage grouting test, the axial loading pressure is kept unchanged at 3 Megapascal (MPa); the high-pressure nitrogen gas tank is opened, the pressure reducing valve is adjusted to a stable pressure of 0.3 MPa, and then the gas pressure is controlled to be unchanged; the pressure reducing valve and the exhaust valve are closed, the gas inlet valve is opened, the gas pump is started until the pressure in the pressure stabilizing tank reaches the set pressure, and the gas pump is closed; the gas inlet valve is closed, the pressure reducing valve and the mud valve are opened, the pressure stabilizing tank delivers high-pressure gas into the grouting tank, and the grouting pressure is controlled to a stable value of 1 MPa by adjusting the gas inlet valve and the mud valve, then the grouting pressure is controlled to be unchanged.

By measuring the changes of grout flow rate and gas flow rate during the grouting process; during this process, the flow meters at the grouting port and gas inlet, and the pressure sensors at the grouting port, gas inlet and gas outlet respectively monitor the grout flow rate, gas inlet flow rate, gas pressure at the gas inlet, and gas pressure at the gas outlet, and feed them back to the data acquisition instrument. After computer processing, the permeability change line graph shown in FIG. 4 is obtained.

### Embodiment 4

This embodiment is a process of obtaining the nonlinear seepage factor using the device and method of Embodiment 1: combined with the relationship satisfied by the fluid in the rock sample obtained in Embodiment 2:

$$- Δ p = \frac{μ}{k} v + {ρ}_{ω} β {v}^{2} ;$$

On this basis, the nonlinear seepage factor is defined to satisfy the following relationship:

$$F = \frac{{ρ}_{ω} k β v}{μ} ;$$

where μ is the fluid dynamic viscosity coefficient; k is the permeability of the mining-induced fractured rock mass; β is the non-Darcy factor; ρ_{w} is the fluid density; v is the fluid velocity; and p is the fluid pressure.

Substituting the empirical formula of the non-Darcy factor and the porosity formula, the corrected nonlinear seepage factor is obtained as:

$$F = \frac{1.15 \times {10}^{- 6} {ρ}_{w} {h}_{m} {Q}_{{T}_{i}}}{μ ( π {r}^{2} {h}_{m} - {\int }_{0}^{{T}_{i}} Q - \frac{{m}_{s}}{{ρ}_{s}} )} ;$$

where ρ_{w} is the fluid density; mₛ is the mass of the rock sample; ρₛ is the mass density of the rock sample; Q_{Ti} is the gas pipeline flow rate at the gas inlet; r is the bottom radius of the inner grouting cylinder; hₘ is the height of the rock sample after axial pressure loading;

$${\int }_{0}^{{T}_{i}} Q$$

is the cumulative flow rate of the grout pipeline at time Tᵢ; and μ is the fluid dynamic viscosity coefficient.

### Embodiment 5

According to the calculation process of the nonlinear seepage factor of Embodiment 4, a specific application example is provided. Red sandstone is selected as the rock sample for testing, crushed into particles by a rock crusher and screened with a sieve. The particle sizes are selected as a first particle size (0-5 millimeter), second particle size (5-10 millimeters), third particle size (10-15 millimeters), fourth particle size (15-20 millimeters), and fifth particle size (20-25 millimeters). The Talbot index is set to 1, 1200 g of red sandstone is taken with different particle sizes for mixing according to the above Talbot index for characterizing the particle size distribution, then the axial load is loaded to adjust the porosity of the sample, the initial porosity of the sample is set to 0.242, and the height of the sample is set to 140 millimeters. After starting the seepage grouting test, the axial loading pressure is kept unchanged at 3 MPa; the high-pressure nitrogen gas tank is opened, the pressure reducing valve is adjusted to a stable pressure of 0.3 MPa, and then the gas pressure is controlled to be unchanged; the pressure reducing valve and the exhaust valve are closed, the gas inlet valve is opened, the gas pump is started until the pressure in the pressure stabilizing tank reaches the set pressure, and the gas pump is closed. The gas inlet valve is closed, the pressure reducing valve and the mud valve are opened, the pressure stabilizing tank delivers high-pressure gas into the grouting tank, and the grouting pressure is controlled to a stable value of 2 MPa by adjusting the gas inlet valve and the mud valve, then the grouting pressure is controlled to be unchanged.

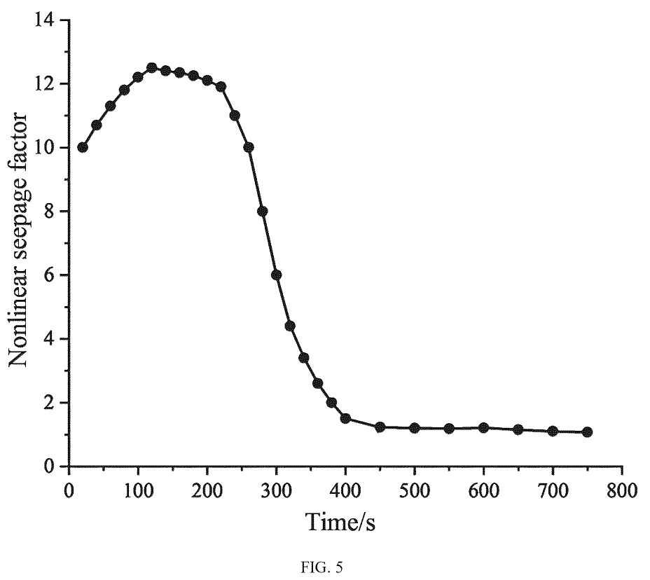

By measuring the changes of grout flow rate and gas flow rate during the grouting process; during this process, the flow meters at the grouting port and gas inlet, and the pressure sensors at the grouting port, gas inlet and gas outlet respectively monitor the grout flow rate, gas inlet flow rate, gas pressure at the gas inlet, and gas pressure at the gas outlet, and feed them back to the data acquisition instrument. After computer processing, the nonlinear seepage factor change line graph shown in FIG. 5 is obtained.

In the description of the present disclosure, it should be understood that the terms “longitudinal”, “transverse”, “upper”, “lower”, “front”, “rear”, “left”, “right”, “vertical”, “horizontal”, “top”, “bottom”, “inner”, “outer” and other indicated orientations or positional relationships are based on the orientations or positional relationships shown in the drawings, which are only for the convenience of describing the present disclosure and do not indicate or imply that the referred device or element must have a specific orientation, be constructed and operated in a specific orientation, and thus may not be understood as a limitation of the present disclosure.

The above-described embodiments are only for describing the preferred modes of the present disclosure, and are not intended to limit the scope of the present disclosure. Without departing from the design spirit of the present disclosure, various modifications and improvements made by those of ordinary skill in the art to the technical solutions of the present disclosure shall fall within the protection scope defined by the claims of the present disclosure.

What is claimed is:

1. A method for grouting test in mining-induced fractured rock masses with real-time synchronous permeability measurement, comprising:
a device for grouting test in mining-induced fractured rock masses with real-time synchronous permeability measurement, comprising:
a base arranged in a pressure applying device, wherein the base is provided with a chamber for storing a sample;
a gas intake part arranged at a bottom of the chamber, wherein a gas outlet end of the gas intake part is in contact with a bottom of the sample, a gas inlet end of the gas intake part is located outside the base, and the gas intake part is configured for communicating with a gas source and injecting high-pressure gas into the chamber;
a gas exhaust part arranged at a top of the chamber, wherein a gas inlet end of the gas exhaust part is in contact with a top of the sample, the gas exhaust part is arranged in a piston, the piston is slidably arranged in the chamber, a gas outlet end of the gas exhaust part is located outside the piston, and a top of the piston is mounted on a pressure applying end of the pressure applying device;
a grouting part wrapped around a side wall of the sample, wherein a bottom of the grouting part is fixedly connected to the base, and a liquid outlet end of the grouting part is in communication with the chamber for injecting grout into the chamber; and
a displacement sensor configured for detecting a height of the piston exceeding the top of the chamber to obtain a height of the sample;
the method further comprising following steps:
filling the sample into the chamber, and controlling the piston to be in contact with the sample;
driving the pressure applying end of the pressure applying device to drive the piston to press down the sample, adjusting a porosity of the sample, and detecting a displacement of the pressure applying end of the pressure applying device through the displacement sensor;
after reaching a target initial porosity, relieving pressure to a stable value, keeping a loading pressure of the pressure applying end of the pressure applying device unchanged, starting the gas source and a grouting mechanism to inject the grout into the chamber, and starting a seepage grouting test;
controlling the grouting part to inject the grout into the grouting mechanism, and enabling the grout to be injected into the chamber;
communicating the gas source with the gas intake part, and controlling the gas source to inject the high-pressure gas into the chamber through the gas intake part;
communicating a gas collection device with the gas exhaust part;
obtaining gas inlet pressure data based on a gas inlet flow rate of the gas intake part;
obtaining gas outlet pressure data based on a gas outlet flow rate of the gas exhaust part;
obtaining grouting pressure data of the grouting part; and
obtaining a permeability and a nonlinear seepage factor based on the gas inlet flow rate, the gas inlet pressure data, the gas outlet flow rate, the gas outlet pressure data, and the grouting pressure data;
wherein a corrected calculation formula of the permeability is:
$$k = \frac{{Q}_{{T}_{i}}}{π {r}^{2} ( {p}_{in} - {p}_{out} )} ( μ + \frac{1.15 \times {10}^{- 6} {ρ}_{w} {h}_{m} {Q}_{{T}_{i}}}{π {r}^{2} {h}_{m} - {\int }_{0}^{{T}_{i}} Q - \frac{{m}_{s}}{{ρ}_{s}}} ) ;$$
wherein pᵢₙ is gas pressure at a gas inlet; pₒᵤₜ is gas pressure at a gas outlet; ρ_{ω} is a fluid density; mₛ is a mass of a rock sample; ρₛ is a mass density of the rock sample; Q_{T}_{_{I}} is a gas pipeline flow rate at the gas inlet; r is a bottom radius of an inner grouting cylinder; hₘ is a height of the rock sample after axial pressure loading;
$${\int }_{0}^{{T}_{i}} Q$$
is a cumulative flow rate of a grout pipeline at time Tᵢ; μ is a fluid dynamic viscosity coefficient; and
wherein a corrected nonlinear seepage factor is:
$$F = \frac{1.15 \times {10}^{- 6} {ρ}_{w} {h}_{m} {Q}_{{T}_{i}}}{μ ( π {r}^{2} {h}_{m} - {\int }_{0}^{{T}_{i}} Q - \frac{{m}_{s}}{{ρ}_{s}} )} ;$$
wherein ρ_{ω} is the fluid density; mₛ is the mass of the rock sample; ρₛ is the mass density of the rock sample; Q_{T}_{_{I}} is the gas pipeline flow rate at the gas inlet; r is the bottom radius of the inner grouting cylinder; hₘ is the height of the rock sample after axial pressure loading;
$${\int }_{0}^{{T}_{i}} Q$$
is the cumulative flow rate of the grout pipeline at time Tᵢ; μ is the fluid dynamic viscosity coefficient.

2. The method for grouting test in mining-induced fractured rock masses with real-time synchronous permeability measurement according to claim 1, wherein the grouting part comprises:
the inner grouting cylinder, wherein a bottom of the inner grouting cylinder is axially connected to the base, the chamber is arranged in the inner grouting cylinder, and a plurality of grouting holes are uniformly formed on a side wall of the inner grouting cylinder for communicating with the chamber;
an outer cylinder coaxially arranged outside the inner grouting cylinder, wherein a bottom of the outer cylinder is fixedly connected to a top of the base, and a top of the outer cylinder is fixedly connected to a top of the inner grouting cylinder;
a grout temporary storage cavity enclosed by an inner wall of the outer cylinder and an outer wall of the inner grouting cylinder, wherein the grout temporary storage cavity is in communication with liquid inlet ends of the plurality of grouting holes; and
a grouting port in communication with the grout temporary storage cavity, wherein the grouting port is formed on the outer cylinder.

3. The method for grouting test in mining-induced fractured rock masses with real-time synchronous permeability measurement according to claim 1, wherein the gas intake part comprises:
a first honeycomb porous gas cavity arranged at the bottom of the chamber, wherein a gas inlet end of the first honeycomb porous gas cavity is in communication with a gas outlet end of a gas intake pipeline, the gas intake pipeline is embedded in the base, a gas inlet of the gas intake pipeline is arranged on a side wall of the base, and the gas inlet is configured for connecting with the gas source; and
a gas-permeable fine gauze laid between a gas outlet end of the first honeycomb porous gas cavity and the sample.

4. The method for grouting test in mining-induced fractured rock masses with real-time synchronous permeability measurement according to claim 1, wherein the gas exhaust part comprises:
a second honeycomb porous gas cavity slidably arranged in the chamber, wherein the second honeycomb porous gas cavity is fixedly connected to a bottom of the piston, a gas inlet end of the second honeycomb porous gas cavity is in contact with and in communication with the top of the sample, a gas outlet end of the second honeycomb porous gas cavity is in communication with a gas inlet end of a gas exhaust pipeline, the gas exhaust pipeline is embedded in the piston, and a gas outlet of the gas exhaust pipeline is formed on a side wall of the piston.

5. The method for grouting test in mining-induced fractured rock masses with real-time synchronous permeability measurement according to claim 2, wherein the displacement sensor comprises:
a linear variable differential transformer, wherein a fixed end of the linear variable differential transformer is fixedly connected to the top of the inner grouting cylinder, and a movable end of the linear variable differential transformer is fixedly connected to the pressure applying end of the pressure applying device.

6. The method for grouting test in mining-induced fractured rock masses with real-time synchronous permeability measurement according to claim 5, wherein the pressure applying device comprises:
an axial displacement control device, wherein the base is arranged in the axial displacement control device, and a movable end of the axial displacement control device is fixedly connected to the top of the piston; and
wherein the movable end of the linear variable differential transformer is fixedly connected to the movable end of the axial displacement control device.
